(19) United States
(12) Patent Application Publication
(10) Pub. No.: US 2023/0407248 A1
(43) Pub. Date: Dec. 21, 2023

(54) METHODS OF INCREASING GROWTH OF CORALS USING A BIOCERAMIC

(71) Applicant: NOVUM CORAL, INC., Goldens Bridge, NY (US)

(72) Inventors: Michael A. DAVITZ, Goldens Bridge, NY (US); William SHEEHAN, Waccabuc, NY (US); Lawrence A. SHIMP, Burlington, WI (US); Onno VISSER, The Hague (NL)

(21) Appl. No.: 18/332,823

(22) Filed: Jun. 12, 2023

Related U.S. Application Data
(60) Provisional application No. 63/351,076, filed on Jun. 10, 2022.
(60) Provisional application No. 63/429,313, filed on Dec. 1, 2022.

(51) Int. Cl. C12N 5/07

(52) U.S. Cl. CPC C12N 5/0601; C12N 2500/05; C12N 2500/14

(57) ABSTRACT

The present disclosure is directed to a method of increasing growth of coral by growing the coral on a bioceramic hydroxyapatite material. An increased growth rate in the coral species is observed with respect to perimeter, surface area and volume as compared with the growth rate on a control material. In some embodiments, the bioceramic hydroxyapatite material contains a carbonatable calcium component with a Ca/P ratio greater than about 1.67.

### CROSS-REFERENCE TO RELATED APPLICATIONS

The present application claims priority to U.S. Provisional Patent Application Nos. U.S. 63/351,076 filed Jun. 10, 2022, and U.S. 63/429,313 filed Dec. 1, 2022.

### FIELD OF THE DISCLOSURE

The present disclosure relates to methods to methods of increasing coral growth using a calcium-containing material.

### BACKGROUND OF THE DISCLOSURE

Coral growth is a complex process. Coral skeletons are composites of 97.5% (w/w) aragonite (CaCO₃), 0.07% (w/w) organics and about 2.5% (w/w) water associated organics. Stony corals form their skeletons by depositing calcium carbonate in the form of aragonite. Corals secrete an organic matrix of proteins locally which facilitates uptake of calcium from sea water. Von Euw et al. Science 356:933-938 (2017). The calcium carbonate then aggregates together in a unique crystal structure called aragonite, which produces the coral skeleton. The skeletal growth of corals consists of two distinct processes: extension (upward growth) and densification (lateral thickening). Gattuso et al. Global and Planetary Change 18 37-46 (1998); Neder et al. Acta Biomaterialia: 96(15):631-645 (2019) and Farfan et al. Coral Reefs (2021). The creation of the coral skeleton is an active biological process where the coral dynamically interacts with its environment. Mass et al. Proc. Natl. Acad. Sci. E7670-E7678 (2017), Zaquin et al. Cryst. Growth Des. 22, 5045-5053 (2022), Khalifa et al. J. Struc. Biol. 213: 107803, and Sun et al. Proc. Natl. Acad. Sci. 117(48):30159-30170 (2020).

Coral reefs are being threatened because of climate change and human activity (pollution and fishing). https://oceanservice.noaa.gov/facts/coralreef-climate.html. Ocean uptake of anthropogenic CO₂ leads to decreased pH, carbonate ion concentration, and saturation state with respect to CaCO₃ minerals, causing increased dissolution of these minerals at the deep seafloor. Sulpis, et al., PNAS, vol. 115, no. 46, pages 11700-11705. Because it reduces the availability of carbonate ions that reef-building corals need to produce their skeletons, ocean acidification (OA) threatens all coral reef ecosystems. Coral calcification rates decline as carbonate ion concentrations decrease.

The commercial trade in coral is another major threat to coral. See Draft report to the USCRTF, https://coralreef.gov/assets/international/trade.pdf.

By 2050, the United Nations estimates that we will lose 70-90% of all coral reefs. https://www.unep.org/news-and-stories/story/why-are-coral-reefs-dying. With the growing global threat to the continued existence of coral reefs, many governments and non-governmental organizations (NGOs) are actively exploring ways to restore coral reefs, including, improving habitat quality for corals, preventing loss of corals and their habitat, enhancing coral population resilience and improving coral health and survival. https://www.fisheries.noaa.gov/national/habitat-conservation/restoring-coral-reefs.

For example, Papke et al. have studied the effect of substrate type on the growth and survival of micro fragments of coral. Papke et al. Frontiers in Marine Science, vol. 8, article 623963.

Hilbertz et al. U.S. Pat. No. 5,543,034 developed a method for the construction, repair and maintenance of corals using an electrochemical system.

The Mote Marine Laboratory has established coral nurseries and explored ways to stimulate coral reproduction. https://mote.org/research/program/coral-reef-restoration.

However, the global threat to corals continues to grow. Thus, there is a continuing need to develop methods and materials for stimulating coral growth and reproduction both in a controlled situation such as a laboratory or coral nursery as well as in the ocean.

### SUMMARY OF THE DISCLOSURE

The methods disclosed provide for a method of increasing growth rate of a coral species, comprising growing the coral species on a bioceramic hydroxyapatite material for a period of time, t. The growth rate can be measured by an increase in the perimeter of the coral species, an increase in surface area of the coral species or an increase in volume of the coral species. The period of time, t, ranges from about 1 month to about 1 year. Other time periods such as 2, 3, 4, 5 6, 7, 8, 9, 10, 11, 12 months, or 1, 2, 3, 4, and 5 years can also be used. In certain embodiments, the growth rate of the coral species on the bioceramic hydroxyapatite material increases from about 10% to about 100% when compared to growth on a control material.

The control material is a ceramic material such as fired clay.

The increase of the perimeter of the coral species ranges from about 0.1 cm/month to 10 cm/month.

The increase of the surface area of the coral species is in the range of from 0.5 cm²/month to 50 cm²/month.

The increase of the volume of the coral species ranges from about 0.25 cm³/month to 20 cm³/month.

The increase in growth rate can be measured by an increase in the amount (% W) of aragonite bioceramic hydroxyapatite block attached to the coral species.

The methods can be used with coral species stoney corals Hexacorallia (large polyp stoney corals (LPS), small poly stoney corals (SPS)) and soft corals (Octocorallia).

The bioceramic hydroxyapatite comprises Ca₁₀(PO₄)₆OH₂ and in certain embodiments comprises a hydroxyapatite material comprising a carbonatable calcium component, said carbonatable calcium component providing said hydroxyapatite material a Ca/P ratio greater than about 1.67.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows an overview of the experimental protocols described in the Example.

FIG. 2a shows the XRD pattern of a bioceramic block as manufactured by CaP Biomaterials, LLC. The calcite XRD diffraction lines are marked. The rest of the diffraction pattern is due to hydroxyapatite. Note that the strongest calcite XRD peak (known as the 100% peak) is located at about 29. 5 degrees 2θ.

FIG. 2b shows the portion of the bioceramic hydroxyapatite block (also referred to as the top) that the coral had attached to. The aragonite diffraction lines are marked. Note that none of these lines were present in figure a. Note also that the calcite 100% peak has significantly diminished in size, along with the other calcite diffraction lines.

FIG. 2c shows a sample of bioceramic hydroxyapatite block at the end of the experiment; this sample is from side of the bioceramic hydroxyapatite block, that is the bottom of the block, not exposed to coral growth. 100% calcite peak at 29.5 degrees 2θ is slightly lower than it was when the block was manufactured, but no aragonite is present.

FIG. 3 shows the perimeter growth of the coral species, Hollywood stunner (Echinopora lamellose) over time exposed to a bioceramic hydroxyapatite block (Green—top line designated) as compared with a control (Orange—lower line designated).

FIG. 4a is a photograph of Digitata montipora mounted on a ceramic control block taken on Jan. 7, 2023.

FIG. 4b is a photograph of Digitata montipora mounted on a bioceramic hydroxyapatite block taken on Jan. 7, 2023.

FIG. 4c is a photograph of Digitata montipora mounted on a ceramic control block taken on Feb. 23, 2023.

FIG. 4d is a photograph of Digitata montipora mounted on a bioceramic hydroxyapatite block taken on Feb. 23, 2023.

FIG. 4e is a photograph of Leptoseris mounted on a ceramic control block taken on Feb. 19, 2023.

FIG. 4f is a photograph of Leptoseris mounted on a bioceramic hydroxyapatite block taken on Feb. 19, 2023.

FIG. 4g is a photograph of Leptoseris mounted on a ceramic control block taken on Feb. 23, 2023.

FIG. 4h is a photograph of Leptoseris mounted on a bioceramic hydroxyapatite block taken on Feb. 23, 2023.

FIG. 4i is a photograph of Echinophyllia aspera mounted on a ceramic control block taken on Feb. 19, 2023.

FIG. 4j is a photograph of Echinophyllia aspera mounted on a bioceramic hydroxyapatite block taken on Feb. 19, 2023.

FIG. 4k is a photograph of Echinophyllia aspera mounted on a ceramic control block taken on Feb. 23, 2023.

FIG. 4l is a photograph of Echinophyllia aspera mounted on a bioceramic hydroxyapatite block taken on Feb. 23, 2023.

FIG. 4m is a photograph of Echinopora lamellosa mounted on a ceramic control block taken on Feb. 19, 2023.

FIG. 4n is a photograph of Echinopora lamellosa mounted on a bioceramic hydroxyapatite block taken on Feb. 19, 2023.

FIG. 4o is a photograph of Echinopora lamellosa mounted on a ceramic control block taken on Feb. 23, 2023.

FIG. 4p is a photograph of Echinopora lamellosa mounted on a bioceramic hydroxyapatite block taken on Feb. 23, 2023.

FIGS. 5a and 5b show the sample photogrammetry photos generated using AgiSoft Metashape (https://www.agisoft.com/) and then viewed in MeshLab (https://www.meshlab.net/#download) of the Leptoseris on the bioceramic hydroxyapatite block (FIG. 5a) and on the ceramic control block (FIG. 5b).

### DETAILED DESCRIPTION OF CERTAIN EMBODIMENTS OF THE DISCLOSURE

The process by which bones and corals form their calcium matrices have some similarities. Helman et al. Proc. Natl. Acad. Sci. 105(1):54-58 (2008), Galow et al. Biochemistry and Biophysics Reports 10: 17-25 (2017), and Mollica et al. Proc. Natl. Acad. Sci. 115(8):1754-1759 (2018). Calcium phosphate materials have been widely used as implant materials in humans and animals. Within this class of calcium phosphates known as orthophosphates, there exists a tremendous diversity of chemical and structural variations. Collectively, these compounds can be referred to as bioceramics which are used for repairing or replacing damaged bone tissues. The most commonly used bioceramics are calcium hydroxyapatite, β-tricalcium phosphate and mixtures of hydroxyapatite and β-tricalcium phosphate (usually referred to as biphasic HA/TCP). All of these materials can be made in a variety of forms, densities or porosities and finished to any shapes or physical characteristics as needed. The bioceramics can also contain biphasic calcium phosphate, calcium carbonate as well as fluorapatite.

The present disclosure provides methods and materials for increasing growth rate of coral, comprising growing a coral fragment or explant on a bioceramic comprising hydroxyapatite or other similar bioceramics as set forth above. The growth rate of the coral can be measured by a variety of different means, including perimeter of the coral, surface area and volume. Formation of aragonite, a crystalline form of calcium carbonate generated by corals, can also be measured.

The methods and materials can be applied to a wide range of different corals, including, the 82 candidate petitioned under the U.S. Endangered Species Act, https://www.fisheries.noaa.gov/resource/document/status-review-report-82-candidate-coral-species-petitioned-under-us-endangered, or the 800 species of reef building corals designated by the United Nations, https://www.unep.org/explore-topics/oceans-seas/what-we-do/protecting-coral-reefs. These corals include both stoney corals (Hexacorallia), such as large polyp stoney corals (LPS) and small poly stoney corals (SPS), as well as soft corals (Octocorallia).

The following is a non-exhaustive list of reef building coral species. https://www.federalregister.gov/documents/2014/09/10/2014-20814/endangered-and-threatened-wildlife-and-plants-final-listing-determinations-on-proposal-to-list-66. The methods disclosed herein can be applied to such reef building species.

Acanthastrea brevis, Acanthastrea hemprichii, Acanthastrea ishigakiensis, Acanthastrea regularis, Acropora aculeus, Acropora acuminata, Acropora aspera, Acropora dendrum, Acropora donei, Acropora globiceps, Acropora horrida, Acropora jacquelineae, Acropora listefi, Acropora lokani, Acropora microclados, Acropora palmerae, Acropora paniculata, Acropora pharaonis, Acropora polystoma, Acropora retusa, Acropora rudis, Acropora speciosa, Acropora striata, Acropora tenella, Acropora vaughani, Acropora verweyi, Agaricia lamarcki, Alveopora allingi, Alveopora fenestrata, Alveopora verrilliana, Anacropora puertogalerae, Anacropora spinosa, Astreopora cucullata, Barabattoia laddi, Caulastrea echinulata, Cyphastrea agassizi, Cyphastrea ocellina, Dendrogyra cylindrus, Dichocoenia stokesii, Euphyllia cristata, Euphyllia paraancora, Euphyllia paradivisa, Galaxea astreata, Heliopora coerulea, Isopora crateriformis, Isopora cuneata, Leptoseris incrustans, Leptoseris yabei, Millepora foveolata, Millepora tuberosa, Montastraea annularis, Montastraea faveolata, Montastraea franksi, Montipora angulata, Montipora australiensis, Montipora calcarea, Montipora caliculata, Montipora dilatata, Montipora flabellata, Montipora lobulata, Montipora patula, Mycetophyllia ferox, Oculina varicosa, Pachyseris rugosa, Pavona bipartita, Pavona cactus, Pavona decussata, Pavona diffluens, Pavona venosa, Pectinia alcicornis, Physogyra lichtensteini, Pocillopora danae, Pocillopora elegans, Porites horizontalata, Porites napopora, Porites nigrescens, Porites pukoensis, Psammocora stellata, Seriatopora aculeata, Turbinaria mesenterina, Turbinaria peltata, Turbinaria remformis, and Turbinaria stellulata.

Other coral species include, Elkhorn Coral (Acropora palmata). Open Brain Coral (Trachyphyllia geoffroyi), Bubble Coral (Plerogyra sinuosa), Staghorn Coral (Acropora eervieornis), Leaf Coral (Pavona decussata), Vase Coral (Montipora capricornis), Venus Sea Fan Coral (Gorgonia flabelluin) or Sun Corals (Tubastraea).

The methods and materials of provide for growth rate of the coral species on the bioceramic materials disclosed herein. The bioceramic material can be in any form, e.g., a powder, microscopic or macroscopic granules or a shaped geometric form such as a square, round, rhomboid, triangular block (any other geometric shape can be used). The term “block” refers to the bioceramic material shaped in a particular form, e.g., cube, rhomboid, cylinder, etc. The blocks can be produced by 3D printing techniques. 3D printing is a form of additive manufacturing technology that fabricates physical objects from model data. During the printing process, models are digitally sliced into 2D layers, the 3D shape is then built from the base upwards through the deposition of melted material. Ruhl E J, Dixson D L (2019) PLoS ONE 14(8).

The bioceramic material can be fixed, attached, sprayed or coated (e.g., dip coated) onto another material such as ceramic material. The ceramic material can be biologically inert. The fixation or attachment process can involve coating with a biocompatible adhesive such as epoxy. Any biocompatible means of adhesion of the bioceramic material to a second surface such as a ceramic can be used.

The methods comprise mounting the coral on the bioceramic material by any biocompatible means, including, using physical means such as clamps, screws or nails, or an adhesive such as epoxy.

Various metals can be added or mixed with the bioceramic material, including, cobalt, cadmium, chromium, copper, iron, manganese, molybdenum, mercury, nickel, silver, thorium, titanium, uranium, vanadium, and zinc, barium, beryllium, calcium, magnesium, radium, and strontium.

Biological growth factors such as fibroblast growth factor (FGF) or other kinase activators or inhibitors, e.g., tyrosine kinase growth factors, may be added to the bioceramic material. Guo et al. Front. Physiol. 2022 https://www.frontiersin.org/articles/10.3389/fphys.2021.759370/full. Both natural and non-natural or synthetic amino acids, vitamins and fatty acids can be added to the bioceramic material. Biocompatible polymers such as poly-L-lactide or poly-D-lactide may also be added to the bioceramic material.

In one embodiment, the bioceramic material comprises hydroxyapatite (also referred to herein as bioceramic hydroxyapatite) in the form of calcium complexed with phosphates (Ca₅OH(PO₄)₃). An example of such bioceramic hydroxyapatite is disclosed in U.S. Pat. Nos. 9,078,955 and 10,016,457, which are incorporated herein in their entireties. Any bioceramic hydroxyapatite can be used with the methods disclosed herein. Fiume et al. Ceramics 2021, 4(4):542-563; https://doi.org/10.3390/ceramics4040039.

The bioceramic hydroxyapatite can be mixed with calcium carbonate to form the calcium-containing substrate. In some embodiments, the bioceramic hydroxyapatite is mixed with TCP. In some embodiments, the ratio of hydroxyapatite:TCP is in the range (% W) of from 5:95 to 95:5, 5:95 to 75:25, 5:95 to 60:40, 5:95 to 40:60, 5:95 to 25:75, 25:75 to 95:5, 25:75 to 75:25, 25:75 to 60:40, 25:75 to 40:60, 40:60 to 95:5, 40:60 to 75:25, 40:60 to 60:40, 60:40 to 60:40 to 75:25, or 75:25 to 95:5.

The hydroxyapatite can be formed as a two-phase composite comprising a hydroxyapatite matrix phase and a discontinuous phase within said matrix phase, said composite comprising a Ca/P ratio greater than about 1.67, said discontinuous phase comprising a plurality of elongated, randomly-oriented calcium carbonate inclusions having a length dimension of about 5 microns to about 20 microns, said calcium of said inclusions the excess calcium portion of said Ca/P ratio. In some embodiments, at least about 90% of said inclusions have a cross-dimension less than about 10 microns. In some embodiments, the hydroxyapatite is sintered, while in other embodiments, the hydroxyapatite comprises granulated morphology.

The hydroxyapatite in the two-phase composite comprises a matrix phase comprising a sintered calcium phosphate component and a discontinuous phase within such a matrix phase, such a discontinuous phase comprising a plurality of elongated carbonate inclusions. In certain embodiments, such a calcium phosphate component can be selected from sintered hydroxyapatite materials with a Ca/P ratio equal to about or greater than about 1.67. In certain such embodiments, an amount of excess calcium, equal to about 10% to about 25% or more of the total amount of calcium contained in the hydroxyapatite phase can be calcium carbonate. Alternatively, in certain such embodiments, about 15% to about 20% of such a composition can be calcium carbonate. The remainder of any excess calcium not calcium carbonate can be in the form of a non-carbonate salt of calcium such as but not limited to calcium oxide, calcium hydroxide, or a calcium salt other than calcium carbonate.

The hydroxyapatite can have a non-powder, granulate morphology—whether porous or non-porous. In certain such porous embodiments, a pore of such a composition can have a cross-dimension of about 50 microns to about 2000 microns (100 microns-1500 microns, 200 microns-1000 microns, 200 microns-600 microns, 500-900 microns).

The hydroxyapatite can be formed by a method that comprises providing a hydroxyapatite material comprising a carbonatable calcium component, said carbonatable calcium component providing said hydroxyapatite material a Ca/P ratio greater than about 1.67; sintering said hydroxyapatite material; and treating said sintered hydroxyapatite material with a carbon dioxide source to convert said carbonatable calcium component to a discontinuous calcium carbonate phase within a hydroxyapatite phase, said discontinuous phase comprising a plurality of elongated calcium carbonate inclusions, said conversion providing the excess calcium portion of said Ca/P ratio as said elongated calcium carbonate inclusions.

The hydroxyapatite comprises an extraneous carbonatable calcium component. In some methods of preparing the hydroxyapatite, the extraneous carbonatable calcium component is selected from CaO and a calcium oxide precursor selected from Ca(OH)₂, CaCO₃, Ca(NO₃)₂, CaSO₄ and calcium salts of organic acids, and combinations of said calcium oxide and said calcium oxide precursors. The hydroxyapatite material can sintered at a temperature less than 1,200° C. Where more carbonate conversion with less sintering, the hydroxyapatite can be partially sintered.

In some embodiments, the hydroxyapatite can be form by a method that comprises providing a hydroxyapatite material comprising a carbonatable calcium component, said carbonatable calcium component providing said hydroxyapatite material a Ca/P ratio greater than about 1.67; sintering said hydroxyapatite material; exposing said sintered hydroxyapatite material to boiling water; and treating said sintered hydroxyapatite material with a carbon dioxide source to convert said carbonatable calcium component to provide a composition comprising a discontinuous calcium carbonate phase within a hydroxyapatite phase, said discontinuous phase comprising a plurality of elongated calcium carbonate inclusions, said conversion providing the excess calcium portion of said Ca/P ratio as said elongated calcium carbonate inclusions. The carbon dioxide used can be in gaseous and liquid states.

The growth rate of the coral is measured by an increase in the perimeter of the coral fragment to be tested over a time interval t. As used herein, lower case “t” when used separately refers to any designated time period.

In one embodiment, the perimeter growth of the coral when exposed to the bioceramic material is in the range of from 0.1 cm/month to 10 cm/month, 0.1 cm/month to 9 cm/month, 0.1 cm/month to 8 cm/month, 0.1 cm/month to 7 cm/month, 0.1 cm/month to 6 cm/month, 0.1 cm/month to 5 cm/month, 0.1 cm/month to 4 cm/month, 0.1 cm/month to 3 cm/month, 0.1 cm/month to 2 cm/month, 0.1 cm/month to 1 cm/month, 0.1 cm/month to 0.5 cm/month, 0.5 cm/month to 10 cm/month, 0.5 cm/month to 9 cm/month, 0.5 cm/month to 8 cm/month, 0.5 cm/month to 7 cm/month, 0.5 cm/month to 6 cm/month, 0.5 cm/month to 5 cm/month, 0.5 cm/month to 4 cm/month, 0.5 cm/month to 3 cm/month, 0.5 cm/month to 2 cm/month, 0.5 cm/month to 1 cm/month, 1 cm/month to 10 cm/month, 1 cm/month to 9 cm/month, 1 cm/month to 8 cm/month, 1 cm/month to 7 cm/month, 1 cm/month to 6 cm/month, 1 cm/month to 5 cm/month, 1 cm/month to 4 cm/month, 1 cm/month to 3 cm/month, 1 cm/month to 2 cm/month, 2 cm/month to 10 cm/month, 2 cm/month to 9 cm/month, 2 cm/month to 8 cm/month, 2 cm/month to 7 cm/month, 2 cm/month to 6 cm/month, 2 cm/month to 5 cm/month, 2 cm/month to 4 cm/month, 2 cm/month to 3 cm/month, 3 cm/month to 10 cm/month, 3 cm/month to 9 cm/month, 3 cm/month to 8 cm/month, 3 cm/month to 7 cm/month, 3 cm/month to 6 cm/month, 3 cm/month to 5 cm/month, 3 cm/month to 4 cm/month, 4 cm/month to 10 cm/month, 4 cm/month to 9 cm/month, 4 cm/month to 8 cm/month, 4 cm/month to 7 cm/month, 4 cm/month to 6 cm/month, 4 cm/month to 5 cm/month, 5 cm/month to 10 cm/month, 5 cm/month to 9 cm/month, 5 cm/month to 4 cm/month, 5 cm/month to 7 cm/month, 5 cm/month to 6 cm/month, 6 cm/month to 10 cm/month, 6 cm/month to 9 cm/month, 6 cm/month to 8 cm/month, 6 cm/month to 7 cm/month, 7 cm/month to 10 cm/month, 7 cm/month to 9 cm/month, 7 cm/month to 8 cm/month, 8 cm/month to 10 cm/month, 8 cm/month to 9 cm/month, or 9 cm/month to 10 cm/month. Other time periods such as 2, 3, 4, 5 6, 7, 8, 9, 10, 11, 12 months, or 1, 2, 3, 4, and 5 years can also be used.

In another embodiment, the growth rate of coral when exposed to the bioceramic material relative to a control such as a ceramic over a time interval t shows the following increases, from 1.25 times to 5 times, 1.25 times to 4 times, 1.25 times to 3 times, 1.25 times to 2 times, 1.25 times to 1.5 times, 1.5 times to 5 times, 1.5 times to 4 times, 1.5 times to 3 times, 1.5 times to 2 times, 2 times to 5 times, 2 times to 4 times, 2 times to 3 times, 3 times to 5 times, 3 times to 4 times, or 4 times to 5 times. In some embodiments, the growth of coral is measured by an increase in the perimeter of the coral that occurs during a period of time, t as set forth above.

The growth rate of the coral perimeter of the coral when exposed to the bioceramic material is increased relative to a control by greater than about 20% within a 1 month period. Increases in perimeter growth can include from 20%-100%, 20%-80%, 20%-60%, 20%-40%, 40%-100%, 40%-80%, 40%-60%, 60%-100%, 60%-80%, or 80%-100% within a 1 month period. Other time periods such as 2, 3, 4, 5 6, 7, 8, 9, 10, 11, 12 months, or 1, 2, 3, 4, and 5 years can also be used.

The growth rate of the coral when exposed to the bioceramic material can be measured by an increase in the surface area of the coral over a time interval t. In some embodiments, the increase of the surface area growth of the coral when exposed to the bioceramic material is in the range of from 0.5 cm²/month to 50 cm²/month, 0.5 cm²/month to 30 cm²/month, 0.5 cm²/month to 10 cm²/month, 0.5 cm²/month to 5 cm²/month, 0.5 cm²/month to 1 cm²/month, 1 cm²/month to 50 cm²/month, 1 cm²/month to 30 cm²/month, 1 cm²/month to 10 cm²/month, 1 cm²/month to 5 cm²/month, 5 cm²/month to 50 cm²/month, 5 cm²/month to 30 cm²/month, 5 cm²/month to 10 cm²/month, 10 cm²/month to 50 cm²/month, 10 cm²/month to 30 cm²/month, or 30 cm²/month to 50 cm²/month. Other time periods such as 2, 3, 4, 5 6, 7, 8, 9, 10, 11, 12 months, or 1, 2, 3, 4, and 5 years can also be used.

In another embodiment, the growth rate of coral when exposed to the bioceramic material is measured by an increase in the surface area of the coral relative to a ceramic control over a time interval t such as 1 month. In some embodiments, the increase of the surface area growth of the coral relative to a control is in the range of from 1.25 times to 5 times, 1.25 times to 4 times, 1.25 times to 3 times, 1.25 times to 2 times, 1.25 times to 1.5 times, 1.5 times to 5 times, 1.5 times to 4 times, 1.5 times to 3 times, 1.5 times to 2 times, 2 times to 5 times, 2 times to 4 times, 2 times to 3 times, 3 times to 5 times, 3 times to 4 times, or 4 times to 5 times. Other time periods such as 2, 3, 4, 5 6, 7, 8, 9, 10, 11, 12 months, or 1, 2, 3, 4, and 5 years can also be used.

In one embodiment, the surface area of the coral exposed to the bioceramic material is increased an amount relative to a ceramic control in the range of from 20%-100%, 20%-80%, 20%-60%, 20%-40%, 40%-100%, 40%-80%, 40%-60%, 60%-100%, 60%-80%, or 80%-100% within a 1 month period. Other time periods such as 2, 3, 4, 5 6, 7, 8, 9, 10, 11, 12 months, or 1, 2, 3, 4, and 5 years can also be used.

In another embodiment, the growth of coral when exposed to the bioercamic material is measured by an increase in the volume of the coral over a time interval t. In some embodiments, the increase of the volume growth of the coral is in the range of from 0.25 cm³/month to 20 cm³/month, 0.25 cm³/month to 15 cm³/month, 0.25 cm³/month to 10 cm³/month, 0.25 cm³/month to 5 cm³/month, 0.25 cm³/month to 2 cm³/month, 0.25 cm³/month to 1 cm³/month, 1 cm³/month to 20 cm³/month, 1 cm³/month to 15 cm³/month, 1 cm³/month to 10 cm³/month, 1 cm³/month to 5 cm³/month, 1 cm³/month to 2 cm³/month, 2 cm³/month to 20 cm³/month, 2 cm³/month to 15 cm³/month, 2 cm³/month to 10 cm³/month, 2 cm³/month to 5 cm³/month, 5 cm³/month to 20 cm³/month, 5 cm³/month to 15 cm³/month, 5 cm³/month to 10 cm³/month, 10 cm³/month to 20 cm³/month, 10 cm³/month to 15 cm³/month, or 15 cm³/month to 20 cm³/month. Other time periods such as 2, 3, 4, 5 6, 7, 8, 9, 10, 11, 12 months, or 1, 2, 3, 4, and 5 years can also be used.

The growth of the coral volume when exposed to the bioceramic material is increased relative to a ceramic control by greater than about 20% within a 1 month period. Increases in perimeter growth can include from 20%-100%, 20%-80%, 20%-60%, 20%-40%, 40%-100%, 40%-80%, 40%-60%, 60%-100%, 60%-80%, or 80%-100% within a 1 month period. Other time periods such as 2, 3, 4, 5 6, 7, 8, 9, 10, 11, 12 months, or 1, 2, 3, 4, and 5 years can also be used.

The growth of coral when exposed to the bioceramic material can also be measured by an increase in the amount (% W) of aragonite in the coral exposed to the bioceramic material relative to a ceramic control over a time interval t such as 1 month. The increase in the amount of aragonite (% W) in the coral relative to a control is in the range of from 1.25 times to 5 times, 1.25 times to 4 times, 1.25 times to 3 times, 1.25 times to 2 times, 1.25 times to 1.5 times, 1.5 times to 5 times, 1.5 times to 4 times, 1.5 times to 3 times, 1.5 times to 2 times, 2 times to 5 times, 2 times to 4 times, 2 times to 3 times, 3 times to 5 times, 3 times to 4 times, or 4 times to 5 times. Other time periods such as 2, 3, 4, 5 6, 7, 8, 9, 10, 11, 12 months, or 1, 2, 3, 4, and 5 years can also be used.

### Examples

In the examples that follow, the corals were maintained in a Red Sea Tank (Red Sea USA, Houston, TX). Water conditions were continuously monitored and controlled using a Trident System (Neptune Systems, Morgan Hill, CA) for alkalinity, salinity, temperature, calcium and magnesium.

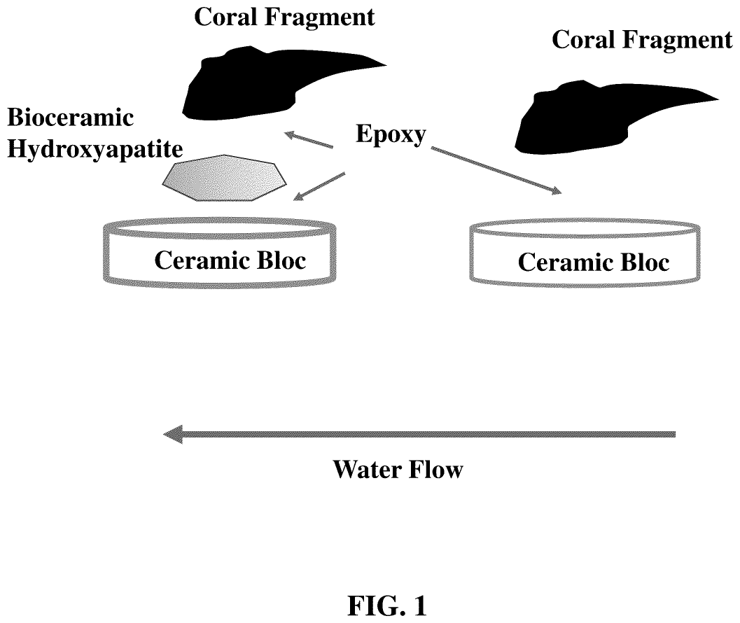

Coral samples were attached with epoxy (Seachem Reef Glue Cyanoacrylate Gel) to a bioceramic hydroxyapatite block obtained from CaP Biomaterials (5-10 grams) which was then affixed directly to a ceramic block (Oceans Wonders LLC, Decorah, IA). Alternatively, the coral samples were affixed directly to a ceramic block (FIG. 1). Coral fragments attached directly to ceramic blocks were used as controls. The ceramic blocks consist of fired clay are biologically inert (Oceans Wonder, Decorah, IA 52101). Control materials can comprise any form of materials that are biologically inert, e.g., fired clay or biologically inactive polymers.

The bioceramic hydroxyapatite blocks comprising hydroxyapatite were obtained from CaP Biomaterials (East Troy, WI 53120). The material has the chemical formula Ca₁₀(PO₄)₆OH₂. The bioceramic hydroxyapatite material was produced in a powder form and then cast into a hexagonal block. The internal surface area of a representative bioceramic hydroxyapatite block was 43.7697 cm² (Particle Technology Labs analysis, Downers Grove, IL).

The control and experimental samples were set-up in the same tank to control for water and lighting conditions. The experimental samples were positioned downstream from the control samples. (FIG. 1).

### Example 1: X-Ray Powder Diffraction (XRD) Particle Analysis

Coral samples were mounted on the bioceramic hydroxyapatite blocks and maintained in the acquarium tanks as described above. A coral fragment of Montipora digitata (also referred to as “Purple Montipora”) was attached with epoxy to a bioceramic hydroxyapatite blocks and the coral left to grow for a period of approximately 4-6 months. After harvest, the coral was physically removed from the hydroxyapatite block. Based on gross physical observation, the coral had physically attached to the block outside of the area containing expoxy.

The bioceramic hydroxyapatite blocks comprises calcium carbonate in the form of calcite which is dispersed in a hydroxyapatite matrix. The portion of the hydroxyapatite block that the coral had been attached to was removed and was examined by XRD analysis. Coral forms calcium carbonated primarily or exclusively in the aragonite crystal form.

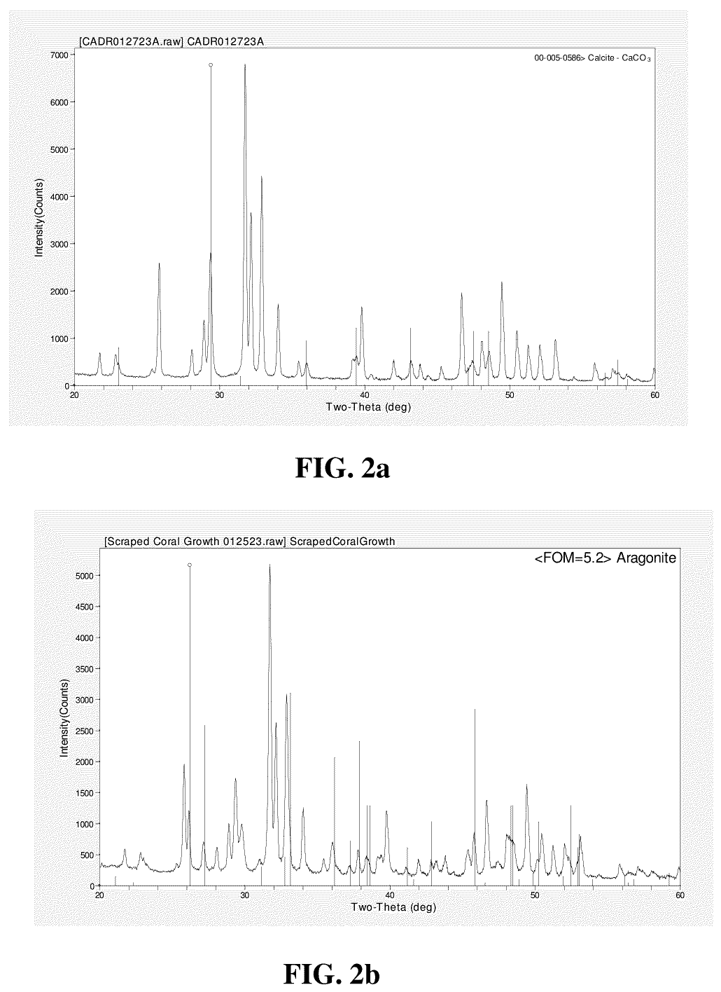

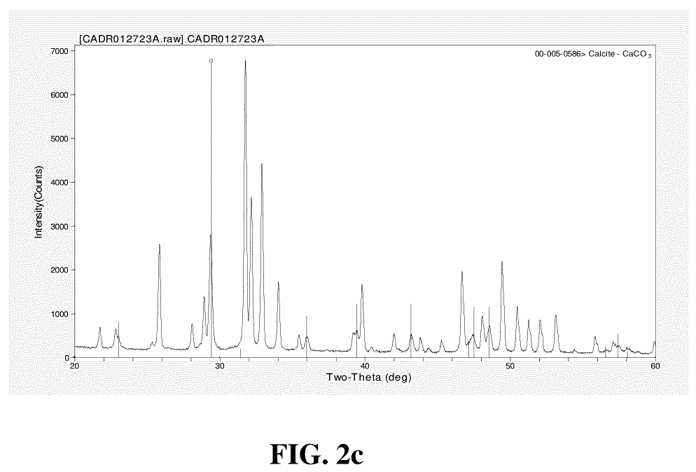

FIG. 2a shows the XRD pattern of a bioceramic block as manufactured by CaP Biomaterials, LLC. The calcite XRD diffraction lines are marked. The rest of the diffraction pattern is due to hydroxyapatite. Note that the strongest calcite XRD peak (known as the 100% peak) is located at about 29. 5 degrees 2θ.

FIG. 2b shows the portion of the bioceramic hydroxyapatite block (also referred to as the top) that the coral had attached to. The aragonite diffraction lines are marked. Note that none of these lines were present in figure a. Note also that the calcite 100% peak has significantly diminished in size, along with the other calcite diffraction lines.

FIG. 2c shows a sample of bioceramic hydroxyapatite block at the end of the experiment; this sample is from side of the bioceramic hydroxyapatite block, that is the bottom of the block, not exposed to coral growth. 100% calcite peak at 29.5 degrees 2θ is slightly lower than it was when the block was manufactured, but no aragonite is present. This diffraction pattern indicates there is slight dissolution of the calcite when exposed to sea water but no conversion to aragonite.

The presence of aragonite indicates that the coral used the calcium from the hydroxyapatite block to form its boney skeleton. Aragonite is a carbonate mineral, a naturally occurring crystal form of calcium carbonate, CaCO₃. It is formed by biological and physical processes, including precipitation from marine and freshwater environments. Aragonite is the high pressure polymorph of calcium carbonate. As such, it occurs in high pressure metamorphic rocks such as those formed at subduction zones. Nesse, William D. (2000). Introduction to mineralogy. New York: Oxford University Press. pp. 336-337.

### Example 2: ICP (Inductively Coupled Plasma) Analysis

Sea water obtained from the ocean was maintained in a polypropylene 1 liter bottle at room temperature. A bioceramic hydroxyapatite block (approximately 4 grams) was added to 1 bottle, while a ceramic block (see above) was added to the second bottle. After 1 month of incubation at room temperature, an ICP analysis (ATI Aquaristik, Hamm Germany) of the sea water was conducted.

TABLE 1
results of ICP analysis of ceramic control block and
hydroxyapatite block in seawater

| | Ceramic Control | Bioceramic Hydroxyapatite |
|---|---|---|
| BASE ELEMENTS | | |
| Salinity | 38.30 PSU | 37.55 PSU |
| Carbonate hardness | 8.41º dKH | 5.89° dKH |
| MAJOR ELEMENTS | | |
| Chloride | 21854 mg/l | 21374 mg/l |
| Sodium | 12091 mg/l | 11929 mg/l |
| Magnesium | 1416 mg/l | 1433 mg/l |
| Sulfur | 976.2 mg/l | 936.5 mg/l |
| Calcium | 455.8 mg/l | 436.1 mg/l |
| Potassium | 457.6 mg/l | 448.3 mg/l |
| Bromine | 79.49 mg/l | 73.97 mg/l |
| Strontium | 9.25 mg/l | 9.06 mg/l |
| Boron | 4.80 mg/l | 4.67 mg/l |
| Fluorine | 1.18 mg/l | 0.76 mg/l |
| MINOR ELEMENTS | | |
| Lithium | 240.4 ug/l | 232.3 ug/l |
| Silicon | 48.26 ug/l | 76.98 ug/l |
| Iodine | 107.9 ug/l | 78.37 ug/l |
| Barium | 22.07 ug/l | 20.64 ug/l |
| Molybdenum | 9.29 ug/1 | 8.92 ug/1 |
| Nickel | n.u. | n.u. |
| Manganese | n.u. | n.u. |
| Arsenic | n.u. | n.u. |
| Beryllium | n.u. | n.u. |
| Chrome | n.u. | n.u. |
| Cobalt | n.u. | n.u. |
| Iron | n.u. | n.u. |
| Copper | 1.20 g/l | 1.43 ug/l |
| Selenium | n.u. | n.u. |
| Silver | n.u. | n.u. |
| Vanadium | 1.79 g/l | n.u. |
| Zinc | 12.50 ug/l | n.u. |
| Tin | 14.57 ug/l | 16.94 ug/l |
| NUTRIENTS | | |
| Nitrate | n.u. | n.u. |
| Phosphorous | 5.73 ug/1 | n.u. |
| Phosphate | 0.02 mg/l | n.u. |
| OTHER | | |
| Aluminum | n.u. | 4.5 ug/l |
| Antimony | n.u. | n.u. |
| Bismuth | n.u. | n.u. |
| Lead | n.u. | n.u. |
| Cadmium | n.u. | n.u. |
| Lanthanum | n.u. | n.u. |
| Thallium | n.u. | n.u. |
| Titanium | n.u. | n.u. |
| Tungsten | n.u. | n.u. |
| Mercury | n.u. | n.u. |

### Example 3: pH Analysis

As part of the dissociation process in seat water, the bioceramic hydroxyapatite releases among other molecules, CaCO₃. Atlas et al. Limnology and Oceanography: V:22(2) 290-300 (1977).

The bioceramic hydroxyapatite blocks were tested for buffering capacity after a CO₂ challenge. A 2-3 gram fragment of the bioceramic hydroxyapatite block was placed in a 50 ml polypropylene tube and sea water added to the tube. As a control, a 2-3 gram fragment of a ceramic block was added to a second tube which was also filled with sea water. The pH of both samples was measured and was 8.07 in each tube. The seawater in each tube was purged with CO₂ by bubbling in CO₂ gas into each tube for approximately 5 minutes. As expected, the resulting pH dropped to about 5.4 in each tube. The pH in each tube was then measured over a 24 hour period. The results are shown in Table 2 below. The pH of the seawater in the tube with the bioceramic hydroxyapatite block sample rose to 8.95, whereas the pH of the seawater in the control tube remained acidic, 6.95.

TABLE 2
pH recovery with CO₂ titration

| | Ceramic | Biocompatible |
|---|---|---|
| Time | Control | Hydroxyapatite |
| Start | 8.07 | 8.07 |
| Immediately after CO₂ purge | 5.41 | 5.46 |
| 1 hour after CO₂ purge | 5.32 | 5.56 |
| 2 hours after CO₂ purge | 6.05 | 5.66 |
| 6-8 hours after CO₂ purge | 6.19 | 7.41 |
| After sitting overnight | 6.95 | 8.95 |

The data indicate that the recovery of the pH values of the seawater was more rapid when exposed to the bioceramic hydroxyapatite blocks.

### Example 4: Coral Perimeter Growth Comparative Analysis

Coral samples were mounted on bioceramic hydroxyapatite blocks and maintained as explained above. Samples of Hollywood stunner (Echinopora lamellose), a well-known LPS plating coral (Hexacorallia), were used to compare the effect of the bioceramic hydroxyapatite block on the growth of the perimeter as compared to a control sample which had been affixed to a ceramic block. (Control, N=4, Experimental N=3. Perimeter growth measured using SketchAndCalc calculator found at https://www.sketchandcalc.com/area-calculator-app/). Plating corals which encompass both SPS and LPS type corals tend to grow horizontally, rather than vertically.

Over a six-month period, the experimental sample (the biocompatible hydroxyapatite block) showed a significant increase of perimeter growth compared to the ceramic control block. FIG. 3 is a plot showing the growth of the perimeter of the coral over time after exposure to the bioceramic hydroxyapatite; the green or upper line shows the growth of perimeter of the coral with the biocompatible hydroxyapatite material, whereas the orange or lower line shows the growth of the perimeter of the coral for the ceramic control samples. A table of perimeter values obtained is shown in Table 3.

TABLE 3
Perimeter values

| | | Ceramic | Bioceramic |
|---|---|---|---|
| Date | | Control | hydroxyapatite |
| 2/10/2022 | Mean | 7.83 cm | 8.56 cm |
| | Standard deviation | 1.03 cm | 0.65 cm |
| 3/18/2022 | Mean | 7.93 cm | 10.04 cm |
| | Standard deviation | 1.08 cm | 0.92 cm |
| 4/6/2022 | Mean | 8.69 cm | 10.55 cm |
| | Standard deviation | 0.70 cm | 0.83 cm |
| 4/17/2022 | Mean | 8.73 cm | 11.02 cm |
| | Standard deviation | 0.73 cm | 0.84 cm |
| 5/8/2022 | Mean | 8.89 cm | 10.79 cm |
| | Standard deviation | 1.12 cm | 1.12 cm |
| 5/21/2022 | Mean | 9.16 cm | 11.21 cm |
| | Standard deviation | 0.57 cm | 1.24 cm |
| 6/27/2022 | Mean | 9.58 cm | 13.02 cm |
| | Standard deviation | 0.57 cm | 1.47 cm |
| 7/26/2022 | Mean | 11.07 cm | 14.17 cm |
| | Standard deviation | 0.77 cm | 1.20 cm |

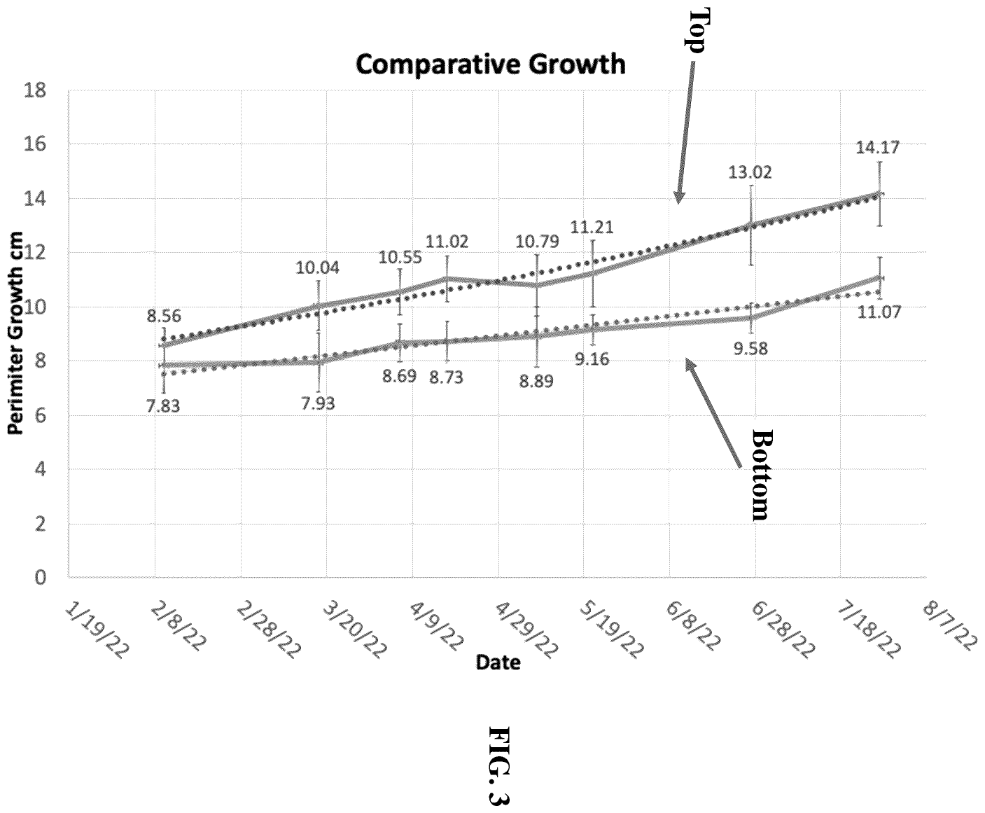

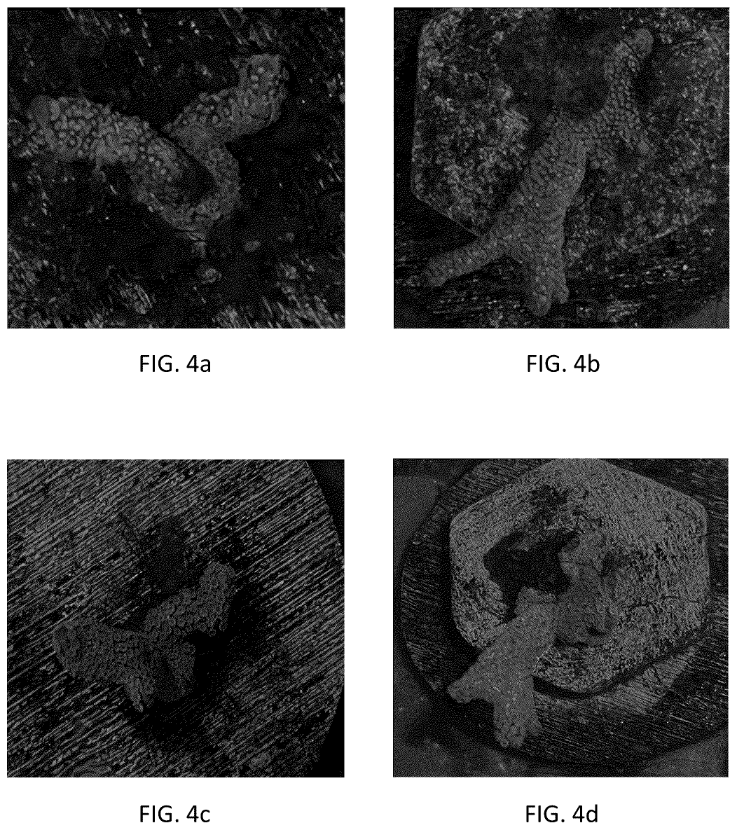

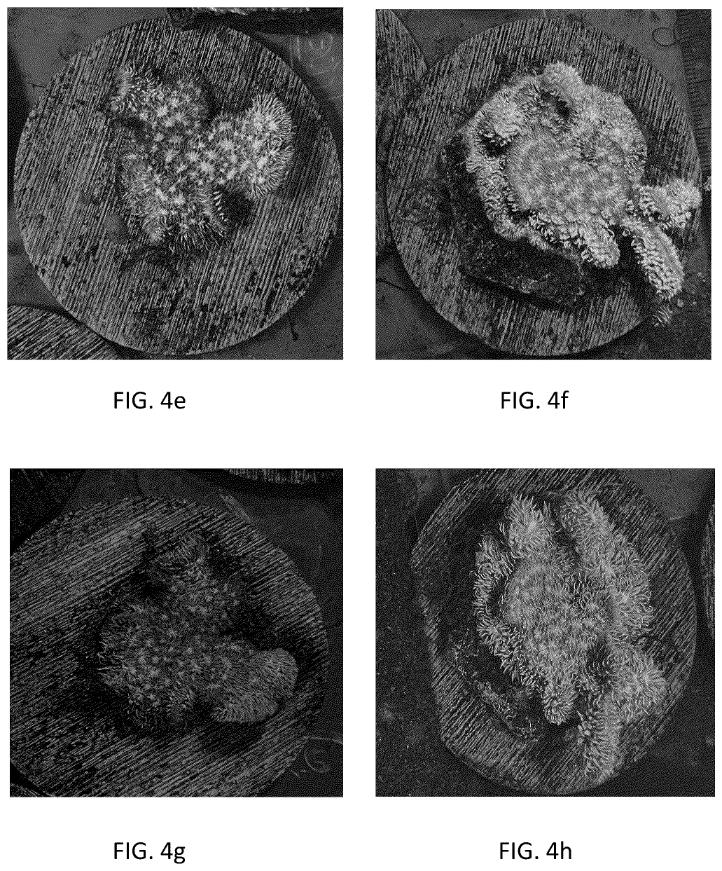

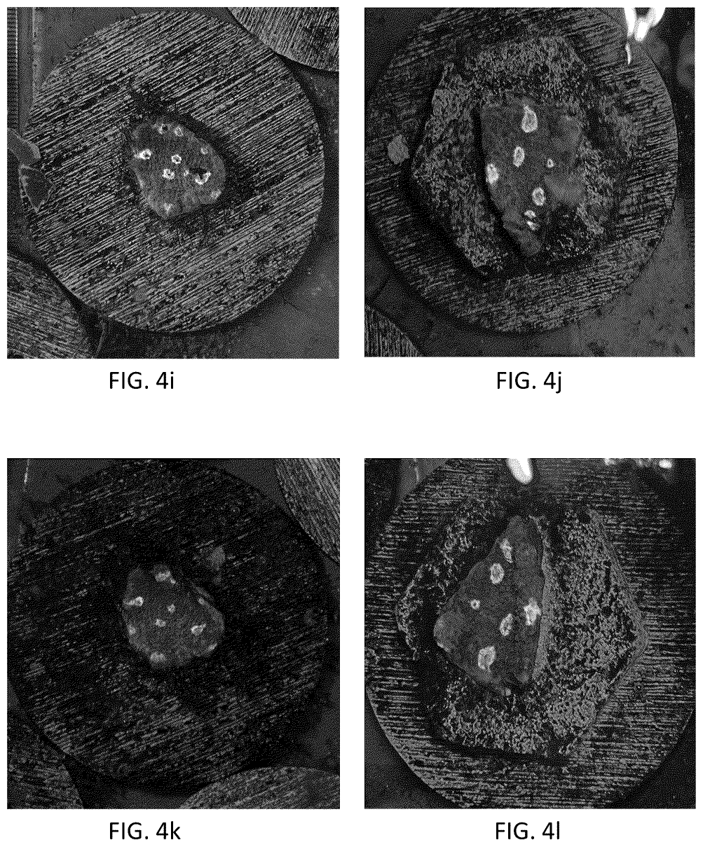

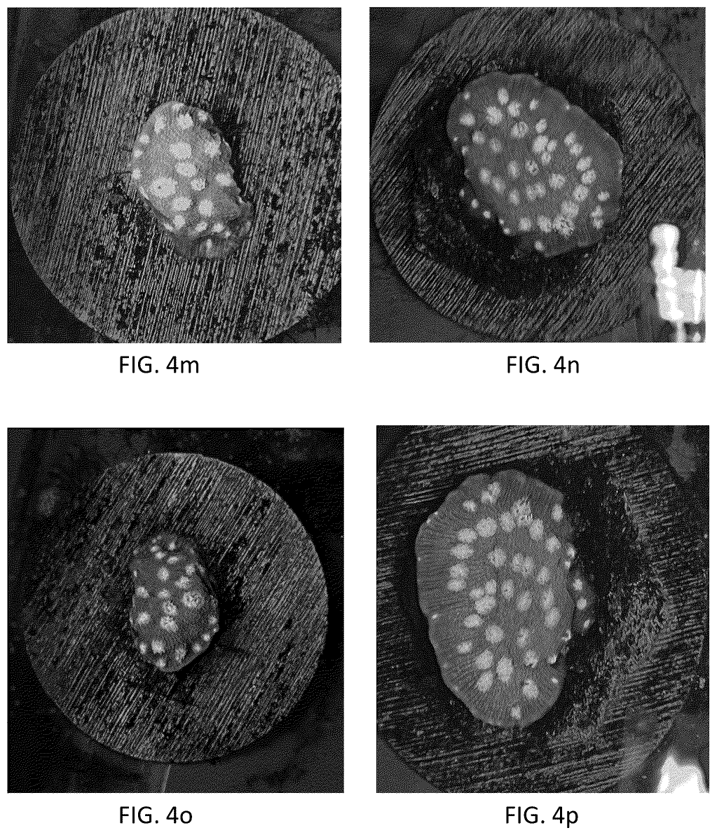

Using Excel, we calculated linear regression statistics for the ceramic control and bioceramic hydroxyapatite samples, including the slope of the regression lines shown in FIG. 3. The slope for regression line for the bioceramic hydroxyapatite sample is 0.031, while the slope for the regression line for the ceramic control is 0.018. Therefore, over the time period, t, shown in FIG. 4, the slope of the bioceramic hydroxyapatite is 1.72× the slope of the ceramic control. We calculated an ANOVA F-Value on the data (9.43) with a p value of 0.008 indicating that there was a significant statistical difference between the bioceramic hydroxyapatite group and the ceramic control group.

### Example 5: Changes in Coral Surface Area Due to Exposure to the Bioceramic Hydroxyapatite Block

Various coral samples were mounted on bioceramic hydroxyapatite blocks or ceramic blocks and maintained as described above. FIGS. 4a-4p are photographs taken at different points in time of various coral mounted on either a bioceramic hydroxyapatite block or a ceramic control block. The list of corals and short description of the figures is provided below.
(a) FIG. 4a is a photograph of Digitata montipora (Hexacorallia), mounted on a ceramic control block taken on Jan. 7, 2023. FIG. 4b is a photograph of Digitata montipora mounted on a bioceramic hydroxyapatite block taken on Jan. 7, 2023.
(b) FIG. 4c is a photograph of Digitata montipora mounted on a ceramic control block taken on Feb. 23, 2023. FIG. 4d is a photograph of Digitata montipora mounted on a bioceramic hydroxyapatite block taken on Feb. 23, 2023.
(c) FIG. 4e is a photograph of Leptoseris (Hexacorallia, SPS) mounted on a ceramic control block taken on Feb. 19, 2023. FIG. 4f is a photograph of Leptoseris mounted on a bioceramic hydroxyapatite block taken on Feb. 19, 2023.
(d) FIG. 4g is a photograph of Leptoseris mounted on a ceramic control block taken on Feb. 23, 2023. FIG. 4h is a photograph of Leptoseris mounted on a bioceramic hydroxyapatite block taken on Feb. 23, 2023.
(e) FIG. 4i is a photograph of Echinophyllia aspera (Hexacorallia, LPS) mounted on a ceramic control block taken on Feb. 19, 2023. FIG. 4j is a photograph of Echinophyllia aspera mounted on a bioceramic hydroxyapatite block taken on Feb. 19, 2023.
(f) FIG. 4k is a photograph of Echinophyllia aspera mounted on a ceramic control block taken on Feb. 23, 2023. FIG. 4i is a photograph of Echinophyllia aspera mounted on a bioceramic hydroxyapatite block taken on Feb. 23, 2023.
(g) FIG. 4m is a photograph of Echinopora lamellosa mounted on a ceramic control block taken on Feb. 19, 2023. FIG. 4n is a photograph of Echinopora lamellosa mounted on a bioceramic hydroxyapatite block taken on Feb. 19, 2023.
(h) FIG. 4o is a photograph of Echinopora lamellosa mounted on a ceramic control block taken on Feb. 23, 2023. FIG. 4p is a photograph of Echinopora lamellosa mounted on a bioceramic hydroxyapatite block taken on Feb. 23, 2023.

It is evident from the photographs that the corals exposed to the bioceramic hydroxyapatite blocks grew at a faster rate than the corals on the ceramic control blocks.

### Example 6: Measure of Coral Surface Area

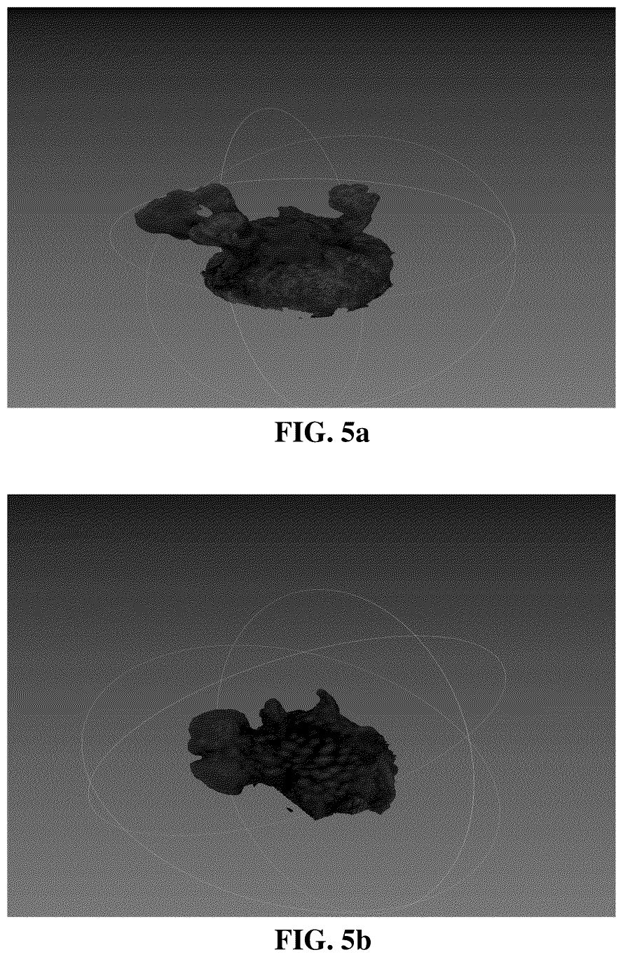

The surface area of the coral samples in Example 5 were measured using photogrammetry. https://oceanexplorer.noaa.gov/technology/photogrammetry/photogrammetry.html. Photogrammetry is a method of approximating a three-dimensional (3D) structure using two dimensional images. Photographs are stitched together using photogrammetry software to make the 3D model and other products like photomosaic maps. It has become an efficient way to rapidly record underwater archaeological sites, and can also be used to characterize seafloor features, such as coral reefs. Million, W. C., & Kenkel, C. D. (2020). Phenotyping 3D coral models in MeshLab vl. https://doi.org/10.17504/protocols.io.bgbpjsmn. FIGS. 5a and 5b show the sample photogrammetry photos generated using AgiSoft Metashape (https://www.agisoft.com/) and then viewed in MeshLab (https://www.meshlab.net/#download) of the Leptoseris on the bioceramic hydroxyapatite block (FIG. 5a) and on the ceramic control block (FIG. 5b). The surface area was obtained using Photogrammetry in MeshLab. Image capture and analysis protocol adapted from Million, W. C., & Kenkel, C. D. (2020). Phenotyping 3D coral models in MeshLab vl. https://doi.org/10.17504/protocols.io.bgbpjsmn.

TABLE 4
Surface Area values

| | | Ceramic | Bioceramic |
|---|---|---|---|
| Species | Date | Control | Hydroxyapatite |
| Digitata montipora | 4/16/2023 | 8.311 cm₂ | 14.948 cm₂ |
| Leptoseris | 4/16/2023 | 4.472 cm₂ | 7.845 cm₂ |
| Echinophyllia aspera | 4/16/2023 | 8.600 cm₂ | 10.302 cm₂ |
| Echinopora lamellosa | 4/16/2023 | 12.484 cm₂ | 28.535 cm₂ |

The data indicate that the surface area of the each of the corals exposed to the bioceramic hydroxyapatite is significantly greater than the surface area of the corals exposed to the ceramic controls. As is evident FIGS. 4a-4p, the starting size of the coral fragments in the ceramic controls as compared with the bioceramic hydroxyapaptite blocks were approximately the same.

It is expected that similar results will be achieved with volumetric calculations.

It will be appreciated by persons skilled in the art that the present disclosure is not limited to what has been particularly shown and described herein above. It is to be understood that the terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “including,” “comprising,” or “having,” “containing,” “involving,” and variations thereof herein, when used in this specification, specify the presence of stated features, integers, steps, operations, elements, and/or components, but do not preclude the presence or addition of one or more other features, integers, steps, operations, elements, components, and/or groups thereof.

Moreover, applicants do not intend for any term in the specification or claims to be ascribed an uncommon or special meaning unless explicitly set forth as such. Further, the present disclosure encompasses present and future known equivalents to the known components referred to herein by way of illustration.

The foregoing description of the specific implementations will so fully reveal the general nature of the disclosure that others can, by applying knowledge within the skill of the relevant art(s), readily modify and/or adapt for various applications such specific implementations, without undue experimentation, without departing from the general concept of the present disclosure. Such adaptations and modifications are therefore intended to be within the meaning and range of equivalents of the disclosed implementations, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one skilled in the relevant art(s).

The subject matter described above is provided by way of illustration only and should not be construed as limiting. Various modifications and changes can be made to the subject matter described herein without following the example embodiments and applications illustrated and described, and without departing from the true spirit and scope of the invention encompassed by the present disclosure, which is defined by the set of recitations in the following claims and by structures and functions or steps which are equivalent to these recitations.

What is claimed is:

1. A method of increasing growth rate of a coral species, comprising growing the coral species on a bioceramic hydroxyapatite material for a period of time, t.

2. The method of claim 1, wherein growth rate is measured by an increase in the perimeter of the coral species.

3. The method of claim 1, wherein growth rate is measured by an increase in surface area of the coral species.

4. The method of claim 1, wherein growth rate is measured by an increase in volume of the coral species.

5. The method of claim 1, wherein the period of time, t, ranges from about 1 month to 1 about year.

6. The method of claim 1, wherein the growth rate of the coral species on the bioceramic hydroxyapatite material increases from about 10% to about 100% when compared to growth on a control material.

7. The method of claim 6, wherein the control material is fired clay.

8. The method of claim 2, wherein increase of the perimeter of the coral species ranges from about 0.1 cm/month to 10 cm/month.

9. The method of claim 8, wherein increase of the surface area of the coral species is in the range of from 0.5 cm²/month to 50 cm²/month.

10. The method of claim 4, wherein the increase of the volume of the coral species ranges from about 0.25 cm³/month to 20 cm³/month.

11. The method of claim 1, wherein the increase in growth rate is measured by an increase in amount (% W) of aragonite in the bioceramic hydroxyapatite material attached to the coral species.

12. The method of claim 1, wherein the coral species comprises stoney corals, Hexacorallia, and soft corals, Octocorallia.

13. The method of claim 12, wherein the coral species comprises stoney corals, Hexacorallia

14. The method of claim 1, wherein the bioceramic hydroxyapatite comprises Ca₁₀(PO₄)₆OH₂.

15. The method of claim 1, wherein the bioceramic hydroxyapatite comprises a hydroxyapatite material comprising a carbonatable calcium component, said carbonatable calcium component providing said hydroxyapatite material a Ca/P ratio greater than about 1.67.
